(19) United States
(12) Patent Application Publication
(10) Pub. No.: US 2007/0015926 A1
(43) Pub. Date: Jan. 18, 2007

(54) Method of preparing R-(+)-alpha-lipoic acid and its salt

(75) Inventors: Xin Chen, Nanjing (CN); Yicheng Mei, Nanjing (CN)

Correspondence Address: Raymond Y. Chan, Suite 128, 108 N. Ynez Avenue, Monterey Park, CA, 91754, US

(73) Assignee: Nanjing Rally Biochemical Inc.

(21) Appl. No.: 11/413,688

(22) Filed: Apr. 28, 2006

(30) Foreign Application Priority Data
Jul. 16, 2005 (CN) 200510081700.9

(51) Int. Cl. C07D 339/04

(52) U.S. Cl. 549/39

(57) ABSTRACT

The present invention discloses a method for preparing R-(+)-LA as well as its metal salts including sodium salt, potassium salt, calcium salt, magnesium salt, zinc salt, ferric salt, cooper salt , lithium salt and its organic salt, wherein a racemic Ethyl 6, 8-Dichlorooctanoic acid is employed as starting agents, followed by a hydrolysis and resolution processes to obtain (+)DCA, and then through sulfuration and cyclization process to obtain R-(+)-LA as well as its varied salts. A method for converting the (−)DCA to (+)DCA is also introduced.

### BACKGROUND OF THE PRESENT INVENTION

1. Field of Invention

The present invention relates organic pharmaceutical products, and more particularly, relates to a method for preparing R-Alpha Lipoic Acid and its Salt.

2. Description of Related Art

R-(+)-α-Lipoic Acid(R-(+)-α-Thioctic Acid, also known R-LA) is one of the enantimoer of racemic α-Lipoic Acid, and is structurally expressed as:

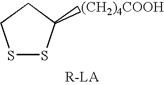

There is plurality of synthesizing process available nowadays for preparing R-LA in practices. Commonly, the process could be categorized into three methods. The first method employs Ethyl 6, 8-Dichloroocanoic acid as initial materials, followed with sulfuration, cyclization, and hydrolysis processes so as to obtain racemic α-Lipoic Acid as disclosed in U.S. Pat. No. 2792406(1957) and U.S. Pat. No. 3,223,712. However, resolving agent has to be used for obtaining R-LA in practice, which results to a merely 45% yield rate. Furthermore, S-Lipoic acid is not prone to be converted into R-Lipoic in practices, there is no doubt such procedure not only waste half the raw materials but also cause soaring costs.

The second process employs Methyl 6, 8-dihydroxyoctanoic acid or Methyl 6-hydroxy-8-chlorooctanoic acid as initiating materials to prepare ester methanesulfonate, to be expanded into R-LA via stereoselectivity as described in U.S. Pat. No. 5869713, G.Bringmann, J.Paust Z.Naturforschung 54b, 661-665, 1999. However, the synthesizing process is complicated and has been struggling to obtain ultimate product with desirable purification.

The third process hydrolyzes racemic Ethyl 6, 8-Dichlorooctanoic acid into (±) dichlorooctanoic acid (DCA), afterwards, (−)ephedrine is employed for salification, resolution processes, and then sulfuration and cyclization processes are subsequently followed to obtain final products(Acker et al, J.Am.Chem.Soc., 76,6483, 1954). Accordingly, such method could save considerable costs, however, around 50% (−)-6, 8-Dichlorooctanoic acid would not be utilized at all.

On the other hand, the intensified pharmacology research had indicated that R-(+)-α-Lipoic Acid had poor heat stability, and susceptible to be polymerized if given adequate oxygen, and be insoluble to the water. Overall, such factors would more or less affect its storage and bioavailability. Therefore, it is desirable to prepare the R-(+)-α-LA into metal-salt form, or organic alkali salt form to improve the stability, solubility as well as the bioavailability for satisfying the demanding request of the medical market. It is foreseeable that (−)-DCA could be converted into (±) DCA then for resolution to obtain (+) DCA, afterwards, sulfuration and cyclization processes would be followed to help generate ultimate R-(+)-α-LA. Unfortunately, there are no such reports or researches been unveiled until now for optimizing the R-(+)-α-LA preparing method.

### SUMMARY OF THE PRESENT INVENTION

A primary object of the present invention is to provide a R-(+)-α-LA preparing method, wherein Ethyl 6, 8-Dichlorooctanoic acid is employed as starting materials to be hydrolyzed into (±)-DCA, after then, followed by racemic resolution by S-(−) α-phenethylamine to obtain (+)DCA, afterwards, sulfuration and cyclization processes are followed to obtain R-(+)-α-LA, mixed with alkali metal or its oxide, halide, and organic alkali to obtain salts, and finally enable (±)DCA be converted into (±)DCA, following by a resolution process to obtain (+)DCA, further by sulfuration and cyclization to synthesize R-(+)-α-LA as well as its salt.

These and other objectives, features, and advantages of the present invention will become apparent from the following detailed description, the accompanying drawings, and the appended claims.

### Brief Description of the Drawings

### DETAILED DESCRIPTION OF THE PREFERRED EMBODIMENT

According to a preferred embodiment of the present invention, (±)DCA is prepared with a resolution process, and then with sulfuration and cyclization processes under a lower temperature, oxygen free, and vacuumed dry condition, so as to synthesize a variety of compounds, such as alkali metal salts as well as organic alkali salts, including sodium salt, potassium salt, calcium salt, magnesium salt, ferric salt, zinc salt, copper salt, lithium salt, N,N-dimethylethanolamine salt, triethylamine salt, trihydroxyethylamine salt, dihydroxyethylethylamine salt, and more particularly, synthesize R-(+)-lipoic potassium salt and R-(+)-lipoic sodium salt through potassium meth, sodium methoxide, potassium ethoxide, and sodium ethoxide.

According to a second embodiment of the present invention, the (−)DCA is converted into (±)DCA by treating the (−)DCA with of lower concentration NaOH, and afterwards, (±)DCA is followed with a resolution process to obtain (+)DCA, to be sulfurized and cyclized into R-(+)-α-LA as well as its salts. The chemical reaction formula is showing below: 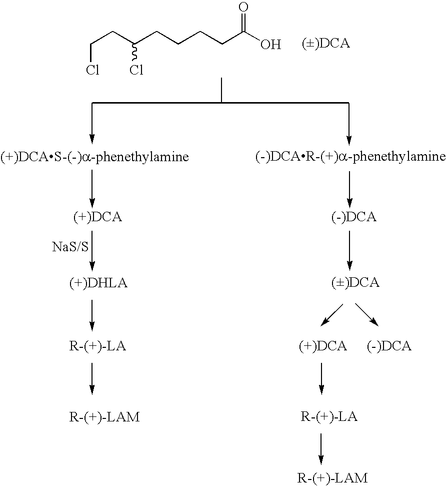 (M=K—Na—Ca—Mg—Zn—Cu—Fe—Li-Organic alkali)

It is noted that Ethyl 6, 8-Dichloroocanoic acid (industrial grade), S-(−)-α-phenethylamine and R-(+)α-phenethylamine(industrial grade) are available in the market.

According to the present invention, the intermediate (±)DCA is through a resolution process first, and then for a synthesizing process to obtain desirable products. Undoubtedly, such procedure would save enormous raw materials and make a solid cost saving. Meanwhile, the final product R-(+)-lipoic acid prepared by the present invention is convenient to transport, storage and application. What is more, the useless (−)DCA could be effectively converted into the (+)DCA for re-utilizing the resources.

Embodiment 1

1, 100 g (0.4mol) racemic Ethyl 6, 8-Dichlorooctanoic acid (95% industrial grade) is prepared first, and then treated with an alkali hydrolysis process so as to obtain (±)-DCA 80 g, to be dissolved into 300 ml ethyl acetate, followed by 24 g (0.2 mol) S-(−)-α-phenethylamine, afterwards, gradually cooled down to a temperature range between 0-10° C., overnight, filtered, and ultimately obtain 30 g wet (+)DCA S-(−)-α-phenethylamine salt.

Afterwards, a recrystallization process is followed, wherein the (+)DCA-S-(−)-α-phenethylamine salt is dissolved in the ethyl acetate, and acidified with diluted hydrochloric acid, and extracted by methylbenzene, and distilled at low temperature to remove solvent, finally obtain 18.8 g (+)-DCA, [α]_{D}²⁵+48-49.6° with a yield rate from 42% to 46%.

2, 18.8 g (+)-DCA is converted into 13.1 g R-(+)-lipoic acid according to a method introduced by U.S. Pat. No. 3,223,712 (1965), with a yield rate of 72%, wherein the melting point is 44-48° C, [α]_{D}²⁵+95-105°.

3, the obtained 10.3 g (0.05 mol) R-(+)-LA (lipoic acid) is dissolved into the 100 ml anhydrous ethanol, and added into sodium ethanol/ethanol solution of same volume, to promptly separate out crystalline solid matter, staying overnight, filtered, vacuumed and dried at room temperature to ultimately obtain 10.8 g white or light yellow colored ultra-fine R-(+)-LA sodium salt with a yield rate of 95%. Compared with the standard R-(+)-LA sodium salt, there is no distinction between rotation, and the purity of HPLC dextro-R-LA is >=98.5%.

Embodiment 2

10.3 g (0.05 mol) R-(+)-LA prepared by the above embodiment 1 is promptly dissolved into 100 ml anhydrous ethanol, and added with same volume of potassium ethanol/ethanol solution, disposed at a temperature range from 0-10° C., to gradually separate out crystal, filtered at a nitrogen-filled condition, vacuumed and dried, and ultimately obtain ultra-fine R-(+)-LA potassium 9.9 g with a yield rate 81%.

Embodiment 3

10.3 g (0.05 mol) R-(+)-LA prepared by the above embodiment 1 is promptly dissolved into 100 ml 95% ethanol, and added with same volume of calcium hydroxide/ethanol suspension, stirred, to separate out sediment, disposed overnight, and filtered. Finally, filtered solid is soaked within 95% ethanol for 72 hours, centrifuged, vacuumed and dried, and ultimately obtain ultra-fine R-(+)-LA calcium 10.2 g with a yield rate 83%.

Embodiment 4

10.3 g (0.05 mol) R-(+)-LA prepared by the above embodiment 1 is promptly dissolved into 100 ml 95% ethanol, and added with same volume of magnesium hydroxide/ethanol suspension, stirred, to separate out sediment, disposed overnight, and filtered. Finally, filtered solid is soaked within 95% ethanol for 72 hours, centrifuged, vacuumed and dried, and ultimately obtain ultra-fine R-(+)-LA magnesium 9.7 g with a yield rate 85%.

Embodiment 5

10.3 g (0.05 mol) R-(+)-LA prepared by the above embodiment 1 is promptly dissolved into 100 ml 95% ethanol, and added with same volume of zinc chloride/ethanol suspension, stirred, to separate out sediment, disposed overnight, and filtered. Finally, filtered solid is soaked within anhydrous ethanol, for 48 hours, repeated, centrifuged, vacuumed and dried, and ultimately obtain ultra-fine R-(+)-LA zinc 10.2 g with a yield rate 89%.

Embodiment 6

10.3 g (0.05 mol) R-(+)-LA prepared by the above embodiment 1 is promptly dissolved into 100ml 95% ethanol, and added with same volume of N,N-dimethylethanolamine/ethanol suspension, stirred, to separate out sediment, cooled down, disposed overnight, and filtered. Finally, filtered solid is soaked within 95% ethanol, centrifuged, vacuumed and dried, and ultimately obtain ultra-fine R-(+)-LA dimethylethanolamine salt 26.0 g with a yield rate of 95%.

Embodiment 7

The mother liquid removed with (+) DCA (mainly (−)DCA) of the above embodiment 1 is added with R-(+) α-phenethylamine, and acidified to obtain (−)DCA, then added with 100 ml ethylene glycol monomethyl ether, and then 5% NaOH water solution, a soared temperature of 60° C., stirred for 5 hours, and the pH value is adjusted to 1-2 by sulfuric acid, extracted by ethyl ether, distilled to remove ethyl ether to obtain (±)DCA, followed by a resolution process to obtain (+) DCA, to be sulfurized and cyclized to obtain R-(+)-LA 9.3 g with a yield rate of 74% to prepare for necessary salts.

One skilled in the art will understand that the embodiment of the present invention as shown in the drawings and described above is exemplary only and not intended to be limiting.

It will thus be seen that the objects of the present invention have been fully and effectively accomplished. Its embodiments have been shown and described for the purposes of illustrating the functional and structural principles of the present invention and is subject to change without departure form such principles. Therefore, this invention includes all modifications encompassed within the spirit and scope of the following claims.

What is claimed is:

1. A method for preparing R-(+)-LA (racemic (+)-lopoic acid), comprising the steps of:
(a) hydrolyzing a racemic Ethyl 6, 8-Dichlorooctanoic acid;
(b) resolving said hydrolyzed racemic Ethyl 6, 8-Dichlorooctanoic acid;
(c) sulfurizing said resolved racemic Ethyl 6, 8-Dichlorooctanoic acid; and
(d) cyclizing said sulfated and resolved racemic Ethyl 6, 8-Dichlorooctanoic acid to obtain said R-(+)-LA.

2. The R-(+)-LA preparing method, as recited in claim 1, wherein the step (b) further comprising a step for utilizing a resolving agent which is selected from a group consisting of S-(−)-α-phenethylamine, R-(+)-α-phenethylamine, and any organic alkali having optical activity.

3. The R-(+)-LA preparing method, as recited in claim 1, further comprising a step for salifying said R-(+)-LA, wherein a salifying materials is selected from a group consisting of alkali metals, alkali metal oxides, alkali metal halide, and alkali metal carbonate, such as potassium ethanol, potassium methanol, sodium ethanol, sodium methanol, calcium hydroxide, magnesium hydroxide, zinc chloride, ferrous chloride, lithium bromide, sodium carbonate, potassium carbonate, and basic cupric carbonate.

4. The R-(+)-LA preparing method, as recited in claim 2, further comprising a step for salifying said R-(+)-LA, wherein a salifying materials is selected from a group consisting of alkali metals, alkali metal oxides, alkali metal halide, and alkali metal carbonate, such as potassium ethanol, potassium methanol, sodium ethanol, sodium methanol, calcium hydroxide, magnesium hydroxide, zinc chloride, ferrous chloride, lithium bromide, sodium carbonate, potassium carbonate, and basic cupric carbonate.

5. The R-(+)-LA preparing method, as recited in claim 1, wherein said R-(+)-LA is salified to potassium salt and sodium salt under a predetermined water-free, nitrogen-filled (oxygen free), low temperature, vacuumed, dried condition for enabling said R-(+)-LA into crystalline monomer thus preventing viscous polymer from creation.

6. The R-(+)-LA preparing method, as recited in claim 2, wherein said R-(+)-LA is salified to potassium salt and sodium salt under a predetermined water-free, nitrogen-filled (oxygen free), low temperature, vacuumed, dried condition for enabling said R-(+)-LA into crystalline monomer thus preventing viscous polymer from creation.

7. The R-(+)-LA preparing method, as recited in claim 3, wherein said R-(+)-LA is salified to potassium salt and sodium salt under a predetermined water-free, nitrogen-filled (oxygen free), low temperature, vacuumed, dried condition for enabling said R-(+)-LA into crystalline monomer thus preventing viscous polymer from creation.

8. The R-(+)-LA preparing method, as recited in claim 4, wherein said R-(+)-LA is salified to potassium salt and sodium salt under a predetermined water-free, nitrogen-filled (oxygen free), low temperature, vacuumed, dried condition for enabling said R-(+)-LA into crystalline monomer thus preventing viscous polymer from creation.

9. The R-(+)-LA preparing method, as recited in claim 1, wherein said racemic Ethyl 6, 8-Dichlorooctanoic acid is dissolved into diluted NaOH and ethylene glycol monomethyl ether, to be converted into (±)DCA.

10. The R-(+)-LA preparing method, as recited in claim 2, wherein said racemic Ethyl 6, 8-Dichlorooctanoic acid is dissolved into diluted NaOH and ethylene glycol monomethyl ether, to be converted into (±)DCA.

11. The R-(+)-LA preparing method, as recited in claim 3, wherein said racemic Ethyl 6, 8-Dichlorooctanoic acid is dissolved into diluted NaOH and ethylene glycol monomethyl ether, to be converted into (±)DCA.

12. The R-(+)-LA preparing method, as recited in claim 4, wherein said racemic Ethyl 6, 8-Dichlorooctanoic acid is dissolved into diluted NaOH and ethylene glycol monomethyl ether, to be converted into (±)DCA.

13. The R-(+)-LA preparing method, as recited in claim 5, wherein said racemic Ethyl 6, 8-Dichlorooctanoic acid is dissolved into diluted NaOH and ethylene glycol monomethyl ether, to be converted into (±)DCA.

14. The R-(+)-LA preparing method, as recited in claim 1, wherein further comprising a step for salifying said R-(+)-LA after said sulfuration and cyclizing step.

15. The R-(+)-LA preparing method, as recited in claim 2, wherein further comprising a step for salifying said R-(+)-LA after said sulfuration and cyclizing step.

16. The R-(+)-LA preparing method, as recited in claim 3, wherein further comprising a step for salifying said R-(+)-LA after said sulfuration and cyclizing step.

17. The R-(+)-LA preparing method, as recited in claim 4, wherein further comprising a step for salifying said R-(+)-LA after said sulfuration and cyclizing step.

18. The R-(+)-LA preparing method, as recited in claim 5, wherein further comprising a step for salifying said R-(+)-LA after said sulfuration and cyclizing step.

19. The R-(+)-LA preparing method, as recited in claim 9, wherein further comprising a step for salifying said R-(+)-LA after said sulfuration and cyclizing step.

20. The R-(+)-LA preparing method, as recited in claim 13, wherein further comprising a step for salifying said R-(+)-LA after said sulfuration and cyclizing step.
